(19) United States
(12) Patent Application Publication
(10) Pub. No.: US 2017/0290754 A1
(43) Pub. Date: Oct. 12, 2017

(54) COMPOSITION COMPRISING A DICARBONYL DERIVATIVE AND METHOD FOR STRAIGHTENING THE HAIR USING THIS COMPOSITION

(71) Applicant: L'OREAL, Paris (FR)

(72) Inventor: Nicolas DAUBRESSE, La Celle Saint Cloud (FR)

(21) Appl. No.: 15/634,271

(22) Filed: Jun. 27, 2017

Related U.S. Application Data
(63) Continuation of application No. 14/441,337, filed on May 7, 2015, now abandoned.

(30) Foreign Application Priority Data
Nov. 9, 2012 (FR) 1360675

(51) Int. Cl. A61K 8/365; A45D 7/06; A45D 2/00; A61K 8/58; A61Q 5/04

(52) U.S. Cl. CPC A61K 8/365; A61K 8/585; A61Q 5/04; A45D 2/001; A45D 7/06

(57) ABSTRACT

The present invention relates to a cosmetic composition comprising:
one or more dicarbonyl derivatives corresponding to formula (I) below, and/or derivatives thereof and/or hydrates thereof and/or salts thereof:

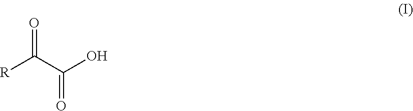

in which formula (I) R=H, COOH, linear or branched C₁-C₆ alkyl which is optionally substituted with an OH or COOH or Br radical; phenyl or benzyl optionally substituted with an OH or COOH radical; or alternatively an indolyl radical or

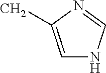

one or more silanes corresponding to formula (II) below and/or oligomers thereof:

R₁Si(OR₂)_{z}(R₃)ₓ(OH)_{y} (II)

in which formula (II):
R₁ is a linear or branched, saturated or unsaturated, cyclic or acyclic C₁-C₆ hydrocarbon-based chain substituted with a group chosen from the following groups:
   amine NH₂ or NHR (R=C₁-C₂₀ and especially C₁-C₆ alkyl optionally substituted with a radical comprising a silicon atom, C₃-C₄₀ cycloalkyl or C₆-C₃₀ aromatic),
   or with a hydroxyl group,
   a thiol group,
   an aryl or aryloxy group which is unsubstituted or substituted, in particular with an amino group or with a C₁-C₄ aminoalkyl group;
   R₁ possibly being interrupted with a heteroatom (O, S or NH) or a carbonyl group (CO),
R₂ and R₃, which may be identical or different, represent a linear or branched alkyl group comprising from 1 to 6 carbon atoms,
y denotes an integer ranging from 0 to 3, and
z denotes an integer ranging from 0 to 3, and
x denotes an integer ranging from 0 to 2,
   with z+x+y=3.

The invention also relates to a process for straightening keratin fibers, especially the hair, using the compounds of formulae (I) and (II) with a straightening step using a straightening iron at a temperature of at least 150° C., preferably ranging from 150 to 250° C.

The present invention relates to a cosmetic composition, especially a hair composition, based on one or more particular dicarbonyl derivatives, and also to a process for straightening keratin fibers, especially the hair, using this composition.

In the hair field, consumers wish to have available compositions which make it possible to introduce a temporary change to their head of hair, while targeting good persistence of the effect produced. In general, it is desired for the change to withstand shampooing for at least fifteen days or even more, depending on the nature of said change.

Treatments already exist for modifying the color or shape of the hair and also, to a certain extent, the texture of the hair. One of the treatments known for modifying the texture of the hair consists in combining heat and a composition comprising formaldehyde. This treatment is especially effective for imparting a better appearance to damaged hair and/or for treating long hair and curly hair.

The action of formaldehyde is associated with its ability to crosslink proteins by reaction on the nucleophilic sites thereof. The heat used may be that of an iron (flat tongs or crimping iron), the temperature of which may generally be up to 200° C. or more. However, it is increasingly sought to avoid the use of such substances, which may prove to be aggressive to the hair and other keratin materials.

Patent application WO 2011/104 282 thus proposed a novel process for semi-permanently straightening the hair, which consists in applying an a-keto acid solution to the hair for 15 to 120 minutes, then drying and, finally, straightening the head of hair with an iron at a temperature of about 200° C. The α-keto acid employed is preferably glyoxylic acid.

However, it has been noted that the use of glyoxylic acid can result in some significant limitations; in particular, at high concentration it may not be well tolerated, in particular when the scalp is sensitive and/or irritated. Its volatility, amplified by the use of heat (iron), can also be a problem. Moreover, cosmetic formulations at acid pH may degrade the hair and/or modify the color thereof.

It is already known practice to use glyoxylic acid esters in hair compositions, in particular in hair dye compositions, as described in document DE19859722, and in reducing compositions, as described in document DE19860239.

However, the efficacy of these compounds is not yet sufficient.

The aim of the invention is to develop a straightening/relaxing composition which is stable over time and which makes it possible to straighten/relax and/or reduce the volume of the hair in an efficient and persistent manner while limiting damage to the hair, while at the same time retaining comfort at the time of application for the user of the composition, but also for the hairdresser who applies it.

Thus, one subject of the present invention is a cosmetic composition comprising:
one or more dicarbonyl compounds corresponding to formula (I) below, and/or derivatives thereof and/or hydrates thereof and/or salts thereof:

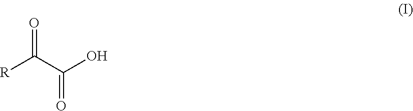

in which formula (I):

R represents an atom or group chosen from i) hydrogen, ii) carboxy —C(O)—OH, iii) linear or branched C₁-C₆ alkyl which is optionally substituted, preferably with at least one hydroxyl —OH radical, carboxy —C(O)—OH radical or halogen such as Br; iv) optionally substituted phenyl, v) optionally substituted benzyl, iv) and v) preferably being optionally substituted with at least one —OH or —C(O)—OH radical; vi) an indolyl radical and vii) an imidazolylmethyl radical and tautomers thereof such as

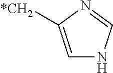

with * representing the part linked to the rest of the molecule,
one or more silanes corresponding to formula (II) below and/or oligomers thereof:

R₁Si(OR₂)_{z}(R₃)ₓ(OH)_{y} (II)

in which formula (II):
R₁ is a linear or branched, saturated or unsaturated, cyclic or acyclic C₁-C₆ hydrocarbon-based chain substituted with a group chosen from the following groups:
   amine NH₂ or NHR (R=C₁-C₂₀ and especially C₁-C₆ alkyl optionally substituted with a radical comprising a silicon atom, C₃-C₄₀ cycloalkyl or C₆-C₃₀ aromatic),
   or with a hydroxyl group,
   a thiol group,
   an aryl or aryloxy group which is unsubstituted or substituted, in particular with an amino group or with a C₁-C₄ aminoalkyl group;
   R₁ possibly being interrupted with a heteroatom (O, S or NH) or a carbonyl group (CO),
R₂ and R₃, which may be identical or different, represent a linear or branched alkyl group comprising from 1 to 6 carbon atoms,
y denotes an integer ranging from 0 to 3, and
z denotes an integer ranging from 0 to 3, and
x denotes an integer ranging from 0 to 2,
   with z+x+y=3.

A subject of the invention is also a process for straightening keratin fibers, especially the hair, using one or more dicarbonyl compounds corresponding to formula (I) and/or derivatives thereof and/or hydrates thereof and/or salts thereof and one or more silanes corresponding to formula (II) and/or oligomers thereof.

In a first variant, the hair straightening process comprises the application to the hair of the composition of the invention, followed by a straightening step using a straightening iron at a temperature of at least 150° C., preferably ranging from 150 to 250° C.

In a second variant, the hair straightening process comprises the successive application to the hair, in any order, with or without intermediate rinsing, of a composition comprising one or more dicarbonyl compounds corresponding to formula (I) and/or derivatives thereof and/or hydrates thereof and/or salts thereof and a composition comprising one or more silanes corresponding to formula (II) and/or oligomers thereof, the application of these compositions being followed by a straightening step using a straightening iron at a temperature of at least 150° C., preferably ranging from 150 to 250° C.

A subject of the invention is also a cosmetic composition that may be obtained by mixing at least two compositions, one comprising at least one silane as described below and the other comprising one or more dicarbonyl compounds corresponding to formula (I) and/or derivatives thereof and/or hydrates thereof and/or salts thereof as described below.

The composition of the invention is stable. The composition of the invention, and the process for treating keratin fibers using it allow good straightening of keratin fibers while limiting damage to these keratin fibers, even when the application of the composition is followed by a heat treatment, in particular using a hair-straightening iron, and have an appreciated wear quality, in particular without excessive vaporization of the composition at the time of straightening. The composition and the process for treating keratin fibers according to the invention also make it possible to limit the change in the color of the fibers and also the problems of breaking of the fibers such as the hair. The composition and the process of the invention will also improve the physical properties of the hair, by durably reducing the frizziness effect of the hair.

In that which follows, the expression “at least one” is equivalent to the expression “one or more”.

Preferably, the composition according to the invention does not comprise any coloring agent or any reducing agent.

According to the present invention, the term “coloring agents” means agents for coloring keratin fibers, such as direct dyes, pigments or oxidation dye precursors (bases and couplers). If they are present, their content does not exceed 0.001% by weight relative to the total weight of the composition. Specifically, at such a content, only the composition would be dyed, i.e. no dyeing effect would be observed on the keratin fibers.

It is recalled that oxidation dye precursors, oxidation bases and couplers are colorless or sparingly colored compounds, which, via a condensation reaction in the presence of an oxidizing agent, give a colored species. With regard to direct dyes, these compounds are colored and have a certain affinity for keratin fibers.

According to the present invention, the term “reducing agent” means an agent that is capable of reducing the disulfide bonds of the hair, such as compounds chosen from thiols, alkaline sulfites, hydrides and phosphines.

In the present invention, the dicarbonyl compounds of formula (I) or derivatives thereof may be in free form, but also in their hydrate forms or in the form of their salts, preferably in free form or in the form of hydrates. As “derivatives” of the dicarbonyl compounds of formula (I), mention may be made of esters of the carboxy group(s), amides of the carboxy group(s), and (thio)acetals and hemi(thio)acetals of the carbonyl function(s) of the compounds of formula (I), in free form or optionally in the form of salts or of hydrates, preferably in free form or in the form of hydrates.

The esters and amides may be synthesized via conventional esterification or amidation processes, from the corresponding acids well known to those skilled in the art.

The esters are obtained, for example, from the acids of formula (I) and from a mono- or polyalcohol.

The term “mono- or polyalcohol” is intended to mean an organic compound comprising one hydroxyl group (monoalcohol) or at least two hydroxyl groups (polyalcohol or polyol); said hydroxylated organic compound possibly being aliphatic, acyclic, linear or branched, or (hetero)cyclic, such as sugars (monosaccharides or polysaccharides) or sugar alcohols.

More particularly, the polyalcohol comprises from 2 to 100 hydroxyl groups, preferentially from 2 to 20 hydroxyl groups, even more preferentially from 2 to 10 hydroxyl groups, and better still 2 or 3 hydroxyl groups.

Preferably, the mono- or polyalcohol is chosen from methanol, ethanol, propanol, isopropanol, butanol, hexanol, ethylene glycol, glycerol, dihydroxyacetone, glucose, sorbitol and menthol.

The amides are obtained, for example, from the acids of formula (I) and from an organic mono- or polyamine.

The term “mono or polyamine” means an organic compound comprising one amino group (monoamine) or at least two (and preferably from 2 to 100, better still from 2 to 20) amine groups; said organic compound possibly being aliphatic, acyclic, linear or branched or (hetero)cyclic.

The term “amino” group means a primary amine group —NH₂, or a secondary amine group >NH.

The mono or polyamine is preferably aliphatic.

This amine is preferably chosen from methylamine, ethylamine, propylamine, isopropylamine, butylamine, hexylamine, monoethanolamine, monopropanolamine, propane-1,2,3-triamine and diaminoacetone.

The (thio)acetals and hemi(thio)acetals of the acids of formula (I) may be obtained, for example, from the reaction of alcohols, for the acetals or hemiacetals, or of thiols, for the thioacetals or hemithioacetals, with blocked forms of the acids, followed by hydrolysis. The alcohols may be the same as those mentioned for the esters. The thiols may be equivalents (referred to as mono- or polythiols) to the mono- or polyalcohols mentioned above, except for the fact that the hydroxyl function(s) of said mono- or polyalcohols are replaced with one or more thiol functions SH of the mono- or polythiols. The acetals or thioacetals may also be cyclic (thio)acetals.

Mention may in particular be made of dimethoxyacetic acid, diethoxyacetic acid, 1,3-dioxane-2-carboxylic acid and 1,3-dioxolane-2-carboxylic acid.

The salts may be salts derived from the interaction of the compounds of formula (I) with acids or bases, it being possible for the acids or bases to be of organic or mineral nature.

Preferably, the salts are salts derived from the interaction of the compounds of formula (I) with bases. Alkali metal or alkaline-earth metal salts, and in particular the sodium salts, will in particular be mentioned.

Preferably, the dicarbonyl derivative(s) corresponding to formula (I) and/or derivatives thereof and/or hydrates thereof and/or salts thereof are chosen from the dicarbonyl derivatives corresponding to formula (I) in which R represents i) a hydrogen atom or ii) a linear or branched C₁-C₆ alkyl group optionally substituted with a carboxy group.

More preferably, they are chosen from glyoxylic acid and pyruvic acid, a derivative thereof and hydrates thereof or salts thereof and more preferentially from glyoxylic acid, derivatives thereof and the hydrate forms of these compounds.

Preferentially, the dicarbonyl compound(s) of formula (I) of the invention are chosen from glyoxylic acid and derivatives thereof and the hydrate forms of these compounds.

Mention may be made of glyoxylic acid and also the hydrate form thereof (HO)₂CH—C(O)—OH, for instance the glyoxylic acid as a 50% aqueous solution sold by the company Merck.

Glyoxylic acid derivatives that may be mentioned include glyoxylic acid esters, glyoxylic acid amides, glyoxylic acid (thio)acetals and hemi(thio)acetals, and glyoxylic acid ester (thio)acetals and hemi(thio)acetals.

The glyoxylic acid esters are obtained, for example, from glyoxylic acid and a mono- or polyalcohol, especially those mentioned previously.

Preferably, the mono- or polyalcohol is chosen from methanol, ethanol, propanol, isopropanol, butanol, hexanol, ethylene glycol, glycerol, dihydroxyacetone, glucose, sorbitol and menthol.

Esters that may in particular be mentioned include methyl glyoxylate, ethyl glyoxylate, glyceryl glyoxylate, dihydroxyacetone glyoxylate, glyceryl diglyoxylate or triglyoxylate, sorbitol mono-, di- or triglyoxylate, glucose mono-, di- or triglyoxylate, menthyl glyoxylate, and the acetals, hemiacetals and hydrates thereof.

The glyoxylic acid amides are, for example, obtained from glyoxylic acid and an organic mono or polyamine.

This amine is preferably chosen from methylamine, ethylamine, propylamine, isopropylamine, butylamine, hexylamine, monoethanolamine, monopropanolamine, propane-1,2,3-triamine and diaminoacetone.

Mention may in particular be made of glyoxylic acid N-β-hydroxyethylamide and glyoxylic acid N-γ-hydroxypropylamide and the (thio)acetals, hemi(thio)acetals and hydrates thereof.

The glyoxylic acid (thio)acetals and hemi(thio)acetals may be obtained, for example, from the reaction of alcohols or thiols, for the thioacetals or hemithioacetals, with blocked forms of glyoxylic acid, followed by hydrolysis. The alcohols may be the same as those mentioned for the esters. The thiols may be equivalents (referred to as mono- or polythiols) to the mono- or polyalcohols mentioned above, except for the fact that the hydroxyl function(s) of said mono- or polyalcohols are replaced with one or more thiol functions SH of the mono- or polythiols. The acetals or thioacetals may also be cyclic (thio)acetals.

According to a particularly preferred embodiment, the compound of formula (I) is glyoxylic acid in hydrate form.

According to one embodiment, the composition of the invention comprises from 0.5% to 15% of one or more dicarbonyl derivatives corresponding to formula (I) and/or of a derivative thereof and/or of hydrate forms thereof and/or salts thereof, preferably from 3% to 15% and preferentially from 5% to 10% by weight relative to the total weight of the composition.

According to the invention, the composition comprises one or more silanes corresponding to formula (II) and/or oligomers thereof.

The term “oligomer” means the polymerization products of the compounds of formula (II) comprising from 2 to 10 silicon atoms.

Preferably, R₂ represents an alkyl group comprising from 1 to 4 carbon atoms, better still a linear alkyl group comprising from 1 to 4 carbon atoms, and preferably the ethyl group.

Preferably, R₃ represents an alkyl group comprising from 1 to 4 carbon atoms, better still a linear alkyl group comprising from 1 to 4 carbon atoms, and preferably methyl or ethyl groups.

Preferably, R₁ is an acyclic chain.

Preferably, the compound of formula (II) comprises only one silicon atom in its structure.

Preferably, R₁ represents an alkyl group, and even more preferentially a linear alkyl group, comprising from 1 to 6 carbon atoms or a C₁-C₆ aminoalkyl group.

Preferably, z ranges from 1 to 3. Even more preferentially, z is equal to 3.

Preferably, R₁ is a linear or branched, saturated or unsaturated C1-C6 hydrocarbon-based chain, substituted with an amine group NH2 or NHR (R=C1-C20 and especially C1-C6 alkyl, C3-C40 cycloalkyl or C6-C30 aromatic).

Preferably, the composition comprises at least one compound of formula (II) chosen from 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane and N-(2-aminoethylaminomethyl)phenethyltrimethoxysilane, better still from 3-aminopropyltriethoxysilane (APTES), 3-aminoethyltriethoxysilane (AETES), 3-aminopropylmethyldiethoxysilane and N-(2-aminoethyl)-3-aminopropyltriethoxysilane or oligomers thereof, and, preferably, the silane is 3-aminopropyltriethoxysilane (APTES) or oligomers thereof.

The silane(s) of formula (II) and/or oligomers thereof may be present in the composition according to the invention in a content ranging from 0.05% to 20% by weight, in particular from 0.1% to 10% by weight and preferably from 0.2% to 5% by weight relative to the total weight of the composition.

Preferably, the weight ratio of dicarbonyl derivatives corresponding to formula (I) and/or a derivative thereof and/or hydrate forms thereof and/or salts/silanes thereof of formula (II) and/or oligomers thereof ranges from 0.1 to 10 and better still from 0.2 to 5.

The composition of the invention may comprise at least one surfactant.

The surfactant(s) may be chosen from nonionic, anionic, cationic and amphoteric or zwitterionic surfactants.

According to a particular embodiment, the composition comprises at least one amphoteric or zwitterionic surfactant.

In particular, the amphoteric or zwitterionic surfactant(s), which are preferably non-silicone, which may be used in the present invention may especially be derivatives of optionally quaternized aliphatic secondary or tertiary amines, in which derivatives the aliphatic group is a linear or branched chain comprising from 8 to 22 carbon atoms, said amine derivatives containing at least one anionic group, for instance a carboxylate, sulfonate, sulfate, phosphate or phosphonate group.

Mention may be made in particular of (C₈-C₂₀)alkylbetaines, (C₈-C₂₀)alkylsulfobetaines, (C₈-C₂₀)alkylamido(C₃-C₈)alkylbetaines and (C₈-C₂₀)alkylamido(C₆-C₈)alkylsulfobetaines.

Among the optionally quaternized secondary or tertiary aliphatic amine derivatives that may be used, as defined above, mention may also be made of the compounds of respective structures (B1) and (B2) below:

Rₐ—(O)—NH—CH₂—CH₂—N⁺(R_{b})(R_{c})—CH₂C(O)O⁻, M⁺, X⁻ (B1)

in which formula:
Rₐ represents a C₁₀-C₃₀ alkyl or alkenyl group derived from an acid Rₐ—COOH preferably present in hydrolyzed copra oil, or a heptyl, nonyl or undecyl group;
R_{b} represents a β-hydroxyethyl group; and
R_{c} represents a carboxymethyl group;
M⁺ represents a cationic counterion derived from an alkali metal or alkaline-earth metal, such as sodium, an ammonium ion or an ion derived from an organic amine, and
X⁻ represents an organic or mineral anionic counterion, such as that chosen from halides, acetates, phosphates, nitrates, (C₁-C₄)alkyl sulfates, (C₁-C₄)alkyl- or (C₁-C₄)alkylaryl sulfonates, in particular methyl sulfate and ethyl sulfate; or alternatively M⁺ and X⁻ are absent;

Rₐ—(O)—NH—CH₂—CH₂—N(B)(B′) (B2)

in which formula:
B represents the group —CH₂—CH₂—O—X′;
B′ represents the group —(CH₂)_{z}Y′, with z=1 or 2;
X′ represents the group —CH₂—C(O)OH, —CH₂—C(O)OZ′, —CH₂—CH₂—C(O)OH or —CH₂—CH₂—C(O)OZ′, or a hydrogen atom;
Y′ represents the group —C(O)OH, —C(O)OZ′, —CH₂—CH(OH)—SO₃H or the group —CH₂—CH(OH)—SO₃—Z′;
Z′ represents a cationic counterion derived from an alkali metal or alkaline-earth metal, such as sodium, an ammonium ion or an ion derived from an organic amine;
R_{a′} represents a C₁₀-C₃₀ alkyl or alkenyl group derived from an acid R_{a′}—C(O)OH, which is preferably present in copra oil or in hydrolyzed linseed oil, an alkyl group, especially a C₁₇ group and its iso form, or an unsaturated C₁₇ group.

The compounds of this type are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Use may also be made of compounds of formula (B′2):

Ra″—NH—CH(Y″)—(CH2)n—C(O)—NH—(CH2)n′—N(Rd)(Re) (B′2)

in which formula:
Y″ represents the group —C(O)OH, —C(O)OZ″, —CH2—CH(OH)—SO3H or the group —CH2—CH(OH)—SO3—Z″;
Rd and Re, independently of each other, represent a C1-C4 alkyl or hydroxyalkyl radical;
Z″ represents a cationic counterion derived from an alkali metal or alkaline-earth metal, such as sodium, an ammonium ion or an ion derived from an organic amine;
Ra″ represents a C10-C30 alkyl or alkenyl group derived from an acid Ra″—C(O)OH, which is preferably present in copra oil or in hydrolyzed linseed oil;
n and n′ denote, independently of each other, an integer ranging from 1 to 3.

Among the compounds of formula (6′2), mention may be made of the compound classified in the CTFA dictionary under the name sodium diethylaminopropyl cocoaspartamide and sold by the company Chimex under the name Chimexane HB.

In accordance with a particular embodiment of the invention, the content of amphoteric or zwitterionic surfactant(s), when they are present, ranges from 0.05% to 30% by weight, preferably from 0.5% to 10% by weight and more preferably from 0.1% to 5% by weight, relative to the total weight of the composition.

The composition according to the invention may also comprise a cellulose-based polymer.

According to the invention, the term “cellulose-based” polymer means any polysaccharide compound having in its structure sequences of glucose residues linked together via β-1,4 bonds; in addition to unsubstituted celluloses, the cellulose derivatives may be anionic, cationic, amphoteric or nonionic. Thus, the cellulose-based polymers of the invention may be chosen from unsubstituted celluloses, including those in a microcrystalline form, and cellulose ethers. Among these cellulose-based polymers, cellulose ethers, cellulose esters and cellulose ester ethers are distinguished. Among the cellulose esters are mineral esters of cellulose (cellulose nitrates, sulfates, phosphates, etc.), organic cellulose esters (cellulose monoacetates, triacetates, amidopropionates, acetatebutyrates, acetatepropionates and acetatetrimellitates, etc.), and mixed organic/mineral esters of cellulose, such as cellulose acetatebutyrate sulfates and cellulose acetatepropionate sulfates. Among the cellulose ester ethers, mention may be made of hydroxypropylmethylcellulose phthalates and ethylcellulose sulfates.

The compositions according to the invention may be in any of the formulation forms conventionally used, and in particular in the form of an aqueous, alcoholic or aqueous-alcoholic, or oily solution or suspension; a solution or a dispersion of the lotion or serum type; an emulsion, in particular of liquid or semi-liquid consistency, of the O/W, W/O or multiple type; a suspension or emulsion of soft consistency of cream (O/W) or (W/O) type; an aqueous or anhydrous gel, or any other cosmetic form.

These compositions may be packaged in pump-action bottles or in aerosol containers, so as to apply the composition in vaporized (lacquer) form or in the form of a mousse. Such packaging forms are indicated, for example, when it is desired to obtain a spray or a mousse, for treating the hair. In these cases, the composition preferably comprises at least one propellant.

The compositions of the invention may be aqueous or anhydrous. They are preferably aqueous and then comprise water at a concentration ranging from 5% to 98%, better still from 5% to 90% and even better still from 10% to 90% by weight relative to the total weight of the composition.

The composition may in particular comprise one or more organic solvents, in particular water-soluble solvents, such as C₁-C₇ alcohols; mention may in particular be made of C₁-C₇ aliphatic monoalcohols, C₆-C₇ aromatic monoalcohols, C₃-C₇ polyols or C₃-C₇ polyol ethers, which may be employed alone or as a mixture with water.

The composition of the invention may also comprise at least one common cosmetic ingredient, chosen in particular from propellants; oils; solid fatty substances and in particular C₈-C₄₀ esters; C₈-C₄₀ acids; C₈-C₄₀ alcohols; sunscreens; moisturizers; antidandruff agents; antioxidants; chelating agents; nacreous agents and opacifiers; plasticizers or coalescers; fillers; silicones and in particular polydimethylsiloxanes;

polymeric or non-polymeric thickeners or gelling agents other than the cellulose-based polymers already mentioned; emulsifiers; polymers, in particular conditioning or styling polymers; fragrances; basifying agents such as sodium hydroxide or acidifying agents; silanes; crosslinking agents. The composition can, of course, comprise several cosmetic ingredients appearing in the above list.

Depending on their nature and the purpose of the composition, the normal cosmetic ingredients can be present in normal amounts which can be easily determined by those skilled in the art and which can be, for each ingredient, between 0.01% and 80% by weight. Those skilled in the art will take care to choose the ingredients included in the composition, and also the amounts thereof, such that they do not harm the properties of the compositions of the present invention.

The pH of the composition is preferably less than 4, and preferentially ranges from 1 to 3, better still from 1.5 to 3 and even better still from 1.7 to 3.

It may be adjusted to the desired value by means of acidifying and/or basifying agents usually used for treating keratin fibers.

The basifying agent may be chosen from mineral or organic or hybrid alkaline agents, or mixtures thereof.

The mineral alkaline agent(s) are preferably chosen from aqueous ammonia, alkali metal carbonates or bicarbonates such as sodium or potassium carbonates and sodium or potassium bicarbonates, sodium hydroxide or potassium hydroxide, or mixtures thereof.

The organic alkaline agent(s) are preferably chosen from organic amines with a pKb at 25° C. of less than 12, preferably less than 10 and even more advantageously less than 6. It should be noted that it is the pKb corresponding to the function of highest basicity.

Mention may be made, as hybrid compounds, of the salts of the abovementioned amines with acids, such as carbonic acid or hydrochloric acid.

The organic alkaline agent(s) are chosen, for example, from amine derivatives such as alkanolamines, oxyethylenated and/or oxypropylenated ethylenediamines, amino acids of amines such as 1,3-diaminopropane, 1,3-diamino-2-propanol, spermine or spermidine.

The term “alkanolamine” means an organic amine comprising a primary, secondary or tertiary amine function, and one or more linear or branched C₁-C₈ alkyl groups bearing one or more hydroxyl radicals.

Sodium hydroxide is in particular suitable for use in the invention.

The acidifying agent may be chosen from mineral or organic acids, for instance hydrochloric acid, phosphoric acid or lactic acid.

The composition according to the invention is preferably in the form of styling or care gels, care lotions or creams, conditioners, masks or sera.

The composition according to the invention may be obtained by mixing at least two compositions, one comprising at least one silane as described below and the other comprising one or more dicarbonyl compounds corresponding to formula (I) and/or derivatives thereof and/or hydrates thereof and/or salts thereof as described below.

A subject of the invention is also a hair straightening process using one or more dicarbonyl compounds corresponding to formula (I) and/or derivatives thereof and/or hydrates thereof and/or salts thereof and one or more silanes corresponding to formula (II) and/or oligomers thereof, with a straightening step using a straightening iron at a temperature of at least 150° C., preferably ranging from 150 to 250° C.

In a first variant, the process of the invention comprises the application of the composition described previously (composition A), followed by a step of straightening the hair with an iron, preferably at a temperature of at least 150° C. The straightening iron is known in the prior art. It consists in straightening the hair with flat heating tongs, which are generally metallic. The straightening irons are generally used at a temperature ranging from 150 to 250° C.

In a second variant, the hair straightening process comprises the successive application to the hair, in any order, with or without intermediate rinsing, of a composition comprising one or more dicarbonyl compounds corresponding to formula (I) and/or derivatives thereof and/or hydrates thereof and/or salts thereof (composition B) and a composition comprising one or more silanes corresponding to formula (II) and/or oligomers thereof (composition C), the application of these compositions being followed by a straightening step using a straightening iron at a temperature of at least 150° C., preferably ranging from 150 to 250° C.

In this variant, it will be preferred to apply composition (C) before composition (B).

The process of the invention may comprise other intermediate steps aimed at improving the straightening of the hair.

According to a particular embodiment, the time of contact of composition (A) or of compositions (B) or (C) on the hair ranges from 10 to 60 minutes and preferably from 20 to 40 minutes. After this or these leave-on time(s), straightening with a brush and with a hairdryer (blow-drying) is performed. The hair is then straightened with a straightening iron at a temperature ranging from 150 to 250° C. and preferably from 210 to 230° C.

The process of the invention may comprise the application of other hair agents as a pretreatment or post-treatment. In particular, it may comprise the application of a conditioning care product as a post-treatment.

According to another embodiment, the hair straightening process comprises a step of washing the hair and then of drying with a hairdryer before applying composition (A) or compositions (B) and (C). According to this particular embodiment, the steps described above are then encountered, such as the contact time of the compositions, the straightening with a straightening iron, the application of a conditioning agent and the rinsing, all these steps possibly being performed independently of each other, blow-drying possibly being intercalated between the contact of the composition according to the invention and the straightening with the iron. According to one particular embodiment, the straightening with the straightening iron is performed in several passes on the hair, in general 8 to 10 passes.

The process of the present invention is preferably performed without a step of permanent reshaping at basic pH or based on a reducing agent.

### EXAMPLES

The following compositions are prepared, compositions 1 and 2 being compositions in accordance with the invention and compositions C1 to C6 being comparative examples.

| | 1 | | C1 | | C2 | | C3 | | 2 | | C4 | |
|---|---|---|---|---|---|---|---|---|---|---|---|---|
| Glyoxylic acid | 5 | g | 5 | g | — | | — | | — | | — | |
| Pyruvic acid | | | — | | — | | — | | 8 | g | 8 | g |
| Aminopropyltriethoxysilane | 5 | g | — | | 5 | g | 5 | g | 5 | g | — | |
| Water | qs 100 | g | qs 100 | g | qs 100 | g | qs 100 | g | qs 100 | g | qs 100 | g |

| | | | | | | |
|---|---|---|---|---|---|---|
| Sodium | qs pH | qs pH | — | qs pH | qs pH | qs pH |
| hydroxide/hydrochloric acid | 2.2 | 2.2 | | 2.2 | 2.2 | 2.2 |

Process for a One-Step Use:

The compositions, optionally shaken before use, are applied at room temperature to curly hair, which may be natural or dyed, or sensitized by a prior bleaching step, at a rate of 1 g per 2 g of hair. After 15 minutes, the hair is rinsed, dried with a hairdryer (blow-drying) and then straightened by treating with flat tongs heated to 210° C. It is subsequently shampooed to examine the persistence of the straightening effects and of modification of the mechanical and cosmetic properties of the fibers.

Compositions C2 and C3 do not make it possible to obtain permanent straightening. Composition 1 makes it possible to obtain straightening properties superior to those obtained with composition C1 and composition 2 makes it possible to obtain straightening properties superior to those obtained with composition C4, whether in terms of curl relaxation, protection of the natural or artificial color, resistance of the fibers to mechanical stresses (pulling, rubbing, twisting), sheen, smooth feel and smooth look.

Process for a Two-Step Use:

According to another process in two steps without rinsing, composition C2 or C3, or alternatively C1, is first applied to hair, at a rate of 1 g per 2 g of hair, the compositions are left to act for 15 minutes and then, without rinsing, 1 g per 2 g of hair of composition C1, or alternatively C2 or C3, is applied for 15 minutes. The hair is then dried (blow-drying) and then straightened by passing through flat tongs brought to 210° C. (10 passes on locks separated into two thicknesses).

The application of composition C1 combined with the application of compositions C2 or C3 leads to performance superior to that obtained with composition C1 used alone.

Similarly, composition C2 or C3, or alternatively C4, is first applied to hair, at a rate of 1 g per 2 g of hair, the compositions are left to act for 15 minutes and then, without rinsing, 1 g per 2 g of hair of composition C4, or alternatively C2 or C3, is applied for 15 minutes. The hair is then dried (blow-drying) and then straightened by passing through flat tongs brought to 210° C. (10 passes on locks separated into two thicknesses).

The application of composition C4 combined with the application of compositions C2 or C3 leads to performance superior to that obtained with composition C4 used alone.

1. A cosmetic composition comprising:
one or more dicarbonyl compounds corresponding to formula (I) below, and/or derivatives thereof and/or hydrates thereof and/or salts thereof: 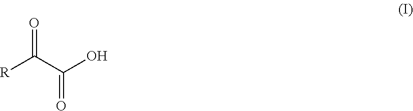
in which formula (I):
R represents an atom or group chosen from i) hydrogen, ii) carboxy —C(O)—OH, iii) linear or branched C₁-C₆ alkyl which is optionally substituted, preferably with at least one hydroxyl —OH radical, carboxy —C(O)—OH radical or halogen such as Br; iv) optionally substituted phenyl, v) optionally substituted benzyl, iv) and v) preferably being optionally substituted with at least one —OH or —C(O)—OH radical; vi) an indolyl radical and vii) an imidazolylmethyl radical and tautomers thereof such as 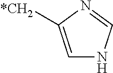
with * representing the part linked to the rest of the molecule,
one or more silanes corresponding to formula (II) below and/or oligomers thereof:
R₁Si(OR₂)_{z}(R₃)ₓ(OH)_{y} (II)
in which formula (II):
R₁ is a linear or branched, saturated or unsaturated, cyclic or acyclic C₁-C₆ hydrocarbon-based chain substituted with a group chosen from the following groups:
amine NH₂ or NHR (R=C₁-C₂₀ and especially C₁-C₆ alkyl optionally substituted with a radical comprising a silicon atom, C₃-C₄₀ cycloalkyl or C₆-C₃₀ aromatic),
or with a hydroxyl group,
a thiol group,
an aryl or aryloxy group which is unsubstituted or substituted, in particular with an amino group or with a C₁-C₄ aminoalkyl group;
R₁ possibly being interrupted with a heteroatom (O, S or NH) or a carbonyl group (CO),
R₂ and R₃, which may be identical or different, represent a linear or branched alkyl group comprising from 1 to 6 carbon atoms,
y denotes an integer ranging from 0 to 3, and
z denotes an integer ranging from 0 to 3, and
x denotes an integer ranging from 0 to 2,
with z+x+y=3.

2. The composition as claimed in claim 1, in which R represents i) a hydrogen atom or ii) a linear or branched C₁-C₆ alkyl group optionally substituted with a carboxyl group.

3. The composition as claimed in either of the preceding claims, in which the dicarbonyl compound(s) corresponding to formula (I) and/or derivatives thereof and/or hydrates thereof and/or salts thereof are chosen from glyoxylic acid and pyruvic acid, a derivative thereof, salts thereof and hydrates thereof, preferably from glyoxylic acid, a derivative thereof and the hydrate forms of these compounds.

4. The composition as claimed in any one of the preceding claims, in which the dicarbonyl derivative(s) corresponding to formula (I) and/or derivatives thereof are chosen from glyoxylic acid esters, glyoxylic acid amides, glyoxylic acid (thio)acetals and hemi(thio)acetals, and glyoxylic acid ester (thio)acetals and hemi(thio)acetals.

5. The composition as claimed in claim 3, in which the glyoxylic acid is in its hydrate form.

6. The composition as claimed in any one of claims 1 to 5, comprising from 0.5% to 15% by weight of one or more dicarbonyl derivatives corresponding to formula (I) and/or derivatives thereof and/or hydrates thereof and/or salts thereof, preferably from 3% to 15% and preferentially from 5% to 10% by weight relative to the total weight of the composition.

7. The composition as claimed in any one of claims 1 to 6, in which R₂ represents an alkyl group comprising from 1 to 4 carbon atoms, better still a linear alkyl group comprising from 1 to 4 carbon atoms, and preferably the ethyl group, and R₃ represents an alkyl group comprising from 1 to 4 carbon atoms, better still a linear alkyl group comprising from 1 to 4 carbon atoms, and preferably methyl or ethyl groups.

8. The composition as claimed in any one of the preceding claims, characterized in that R₁ represents a C₁-C₆ aminoalkyl group.

9. The composition as claimed in any one of the preceding claims, characterized in that z ranges from 1 to 3 and preferably z is equal to 3.

10. The composition as claimed in any one of the preceding claims, characterized in that R₁ is an acyclic chain and in that the compound of formula (II) comprises only one silicon atom in its structure.

11. The composition as claimed in any one of the preceding claims, characterized in that it comprises at least one compound of formula (II) chosen from 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane and N-(2-aminoethylaminomethyl)phenethyltrimethoxysilane, better still from 3-aminopropyltriethoxysilane (APTES), 3-aminoethyltriethoxysilane (AETES), 3-aminopropylmethyldiethoxysilane and N-(2-aminoethyl)-3-aminopropyltriethoxysilane or oligomers thereof.

12. The composition as claimed in any one of the preceding claims, characterized in that it comprises at least one compound chosen from 3-aminopropyltriethoxysilane and oligomers thereof.

13. The composition as claimed in one of the preceding claims, characterized in that the silane(s) of formula (II) and/or the oligomers thereof are present in an active material content ranging from 0.05% to 20% by weight, preferably from 0.1% to 10% by weight, preferably ranging from 0.2% to 20% by weight, relative to the total weight of the composition.

14. The composition as claimed in any one of the preceding claims, in which the weight ratio of dicarbonyl derivatives corresponding to formula (I) and/or a derivative thereof and/or hydrate forms thereof and/or salts/silanes thereof of formula (II) and/or oligomers thereof ranges from 0.1 to 10 and better still from 0.2 to 5.

15. The composition as claimed in any one of the preceding claims, characterized in that it is aqueous and comprises water in a concentration preferably ranging from 5% to 98%, better still from 5% to 90% and even better still from 10% to 90% by weight relative to the total weight of the composition.

16. The composition as claimed in any one of the preceding claims, characterized in that it has a pH of less than 4, the pH preferentially ranging from 1 to 3, better still from 1.5 to 3 and even better still from 1.7 to 3.

17. The composition as claimed in any one of the preceding claims, characterized in that it may be obtained by mixing two compositions, one comprising at least one silane as described in one of claims 1 and 7 to 13 and the other comprising one or more dicarbonyl compounds corresponding to formula (I) and/or derivatives thereof and/or hydrates thereof and/or salts thereof as described in any one of claims 1 to 6.

18. A process for straightening keratin fibers, especially the hair, using one or more dicarbonyl compounds corresponding to formula (I) and/or derivatives thereof and/or hydrates thereof and/or salts thereof as described in any one of claims 1 to 6 and one or more silanes corresponding to formula (II) and/or oligomers thereof as described in one of claims 1 and 7 to 13, with a straightening step using a straightening iron at a temperature of at least 150° C., preferably ranging from 150 to 250° C.

19. The straightening process as claimed in claim 18, which comprises the application to the hair of the composition as claimed in any one of claims 1 to 17, followed by a contact time of between 10 and 60 minutes, followed by a straightening step using a straightening iron at a temperature of at least 150° C., preferably between 150 and 250° C.

20. The straightening process as claimed in claim 18, which comprises the successive application to the hair, in any order, with or without intermediate rinsing, of a composition comprising one or more dicarbonyl compounds corresponding to formula (I) and/or derivatives thereof and/or hydrates thereof and/or salts thereof (composition B) and a composition comprising one or more silanes corresponding to formula (II) and/or oligomers thereof (composition C), the application of these compositions being followed by a time of contact for each of them ranging from 10 to 60 minutes and then a straightening step using a straightening iron at a temperature of at least 150° C., preferably ranging from 150 to 250° C.

21. The use of the composition as claimed in any one of claims 1 to 17, for straightening/relaxing keratin fibers, especially the hair.
